(19) United States
(12) Patent Application Publication
(10) Pub. No.: US 2008/0161260 A1
(43) Pub. Date: Jul. 3, 2008

(54) Methods for Detecting and Quantifying Cell Proliferation In Vivo

(75) Inventors: Zia Rahman, Princeton, NJ (US); Jessica Eden Malberg, Robbinsville, NJ (US); Robert Hamlink Ring, West Windor, NJ (US); Sharon Joy Rosenzweig-Lipson, East Brunswick, NJ (US); Lee E. Schechter, Toms River, NJ (US)

Correspondence Address: PILLSBURY WINTHROP SHAW PITTMAN, LLP, P.O. BOX 10500, MCLEAN, VA, 22102, US

(73) Assignee: Wyeth, Madison, NJ (US)

(21) Appl. No.: 11/844,909

(22) Filed: Aug. 24, 2007

Related U.S. Application Data
(60) Provisional application No. 60/823,401, filed on Aug. 24, 2006.

(51) Int. Cl. A61K 31/70; C12Q 1/68; A61P 43/00

(52) U.S. Cl. 514/44; 435/6

(57) ABSTRACT

The invention provides a rapid, sensitive method for detecting and quantifying in vivo cell proliferation in a mammal.

### RELATED APPLICATIONS

Priority is claimed to U.S. Provisional Application No. 60/823,401, filed Aug. 24, 2006, which is incorporated herein by reference in its entirety.

### BACKGROUND OF THE INVENTION

1. Field of the Invention

This invention relates to investigative and therapeutic methods for detecting and quantifying cell proliferation in vivo in brain and other tissues of mammals.

2. Background of the Invention

There is great interest in developing new methods for inducing and regulating cell proliferation in vivo, in brain, spinal cord, heart, and other vital mammalian tissues, in order to study physiological activities of the tissues, and for therapeutic intervention. For example, it has been shown that exercise and antidepressant medications can increase neurogenesis in adult hippocampus, whereas evidence suggests that a decrease in the proliferation of neurons in the adult hippocampus is linked to major depressive disorder (MDD). There is therefore great interest in detecting and measuring cell proliferation in the hippocampus of research mammals in vivo. A rapid, sensitive method for detecting and measuring cell proliferation is also sought by researchers investigating cell proliferation in laboratory mammals in vivo following spinal cord injury and damage to cardiac tissue. Rapid methods that are currently in use are not sufficiently sensitive to permit detection and quantification of cell proliferation in a single mammal; and sensitive methods such as manual cell counting that can be applied to a single mammal are labor intensive and may take weeks or months to provide a result. Accordingly, there currently is great need for a rapid method for detecting and quantifying cell proliferation in vivo in brain and other mammal tissues with the sensitivity to be used with a single mammal.

5-halo-2′-deoxyuridine (HdU) nucleosides are thymidine analogs that are incorporated into the DNA of dividing cells, and can be used as a marker for cell proliferation. Monoclonal antibodies that bind to HdU can be used to label and detect HdU nucleosides that are incorporated into DNA. Monoclonal antibodies specific for 5-bromo-2′-deoxyuridine (BrdU) that are useful for labeling and detecting BrdU incorporated into DNA are readily available commercially, and BrdU is commonly used for labeling and detecting DNA of proliferating cells in vitro and in vivo. HdU nucleosides such as BrdU are able to cross the blood/brain barrier, and are incorporated into DNA of cells proliferating in the brain. Accordingly, there is interest in using anti-BrdU antibodies to label and detect BrdU-containing DNA of proliferating cells in tissues of the brain. Current methods for quantification of BrdU-positive cells in tissues of laboratory mammals utilize immunohistochemical labeling techniques and manual cell counting methods to detect cells that contain BrdU-labeled DNA. This manual quantification method is labor intensive and challenging, especially for tissue regions in which proliferating cells are clustered and therefore difficult to count. Furthermore, two to three months (or more) may be required for a skilled researcher using such immunohistochemical cell-counting techniques to prepare and analyze tissue sections and microscope slides to detect and quantify cell proliferation in a single tissue sample. The invention described herein satisfies the current need for a rapid, sensitive assay method for detecting and quantifying cell proliferation in vivo in brain and other tissues.

### BRIEF SUMMARY OF THE INVENTION

The invention provides a rapid, sensitive method for detecting and quantifying cell proliferation in a mammal. The method comprises administering an amount of a 5-halo-2′-deoxyuridine (HdU) to a mammal sufficient to effect incorporation of HdU into newly synthesized DNA of proliferating cells in the mammal. Following a time period during which HdU is incorporated by proliferating cells into newly synthesized DNA, a tissue sample is removed from the mammal, and genomic DNA is extracted from cells of the tissue sample. The genomic DNA is then contacted with monoclonal anti-HdU antibodies under conditions in which the antibodies bind to HdU-containing DNA. An enzyme that catalyzes conversion of a substrate to a light-producing product is attached to the anti-HdU antibodies that are bound to the HdU-containing DNA. The enzyme molecules attached to the anti-HdU antibodies that are bound to the HdU-containing DNA are then contacted with a solution containing a substrate compound that is converted by to a light-emitting product, at a sufficient substrate concentration and under conditions such that chemiluminescence is produced, and the intensity of the chemiluminescent signal is determined. The intensity of the chemiluminescence is a quantitative indicator of the amount of HdU-containing DNA in the assay sample.

The assay method of the invention may be performed to detect and quantify cell proliferation in any mammal. For example, the mammal may be a mouse, rat, cat, dog, pig, non-human primate, or human. The mammal that is the subject of the assay method can be of any age or developmental stage; e.g., it can be an embryonic, fetal, infantile, juvenile, adolescent, or adult.

The assay method of the invention can include treating the subject mammal with any physical, chemical, dietary, or pharmacological treatment that stimulates or inhibits cell proliferation in the tissue of interest, or has the potential to do so, prior to administering an HdU, and detecting and quantifying cell proliferation in the tissue, as described above.

The HdU can be administered by any route, including, but not limited to, intraperitoneal, intravenous, intramuscular, and subcutaneous. The method may be performed by administering a dosage of from 25 to 1500 mg/kg of the HdU to the mammal in one day. The HdU may be administered once or several times in one day, and it may be administered on one or multiple days.

The assay method may be performed with any tissue of the subject mammal. For example, the assay can be performed with a tissue sample that comprises at least one tissue selected from blood, bone marrow, pancreas, liver, kidney, intestines, thymus, heart, striated muscle, spinal cord, and brain. Brain tissues that may be used in the assay method include, but are not limited to, the hippocampus and the subventricular zone adjacent to a lateral ventricle. The intensity of the chemiluminescent signal that is generated and detected by the assay method depends on the number of cells in the tissue of interest that divided during the HdU labeling period of the assay method. A chemiluminescent signal above background therefore will only be detected if the tissue that is analyzed contains cells that proliferated during the assay.

5-halo-2′-deoxyuridine nucleosides that are suitable for use in the assay method of the invention, such as 5-bromo-2′-deoxyuridine (BrdU), are readily available commercially. When BrdU is used as the HdU for the assay method, the assay method is performed using monoclonal antibodies that bind specifically to BrdU-containing DNA. Monoclonal anti-BrdU antibodies that are suitable for the invention are also readily available commercially.

The assay method may include contacting the genomic DNA extracted from the tissue of interest with an insoluble support material that is capable of binding to HdU-containing DNA, under conditions in which the HdU-containing DNA binds to the support material. An inner surface of the container in which the assay method is performed may be coated with monoclonal anti-BrdU antibodies, and the genomic DNA is added to the container under conditions such that BrdU-containing DNA contacts and binds to the anti-BrdU antibodies that coat the inner surface of the assay container.

The enzyme that is attached to the anti-HdU antibodies that bind to the HdU-containing DNA may be any enzyme that catalyzes conversion of a substrate into a product that emits light. Examples of enzymes that can be used for the assay method include peroxidase, alkaline phosphatase, β-galactosidase, acid phosphatase, and β-glucuronidase. These enzymes are readily available commercially, as are substrate compounds that are converted by these enzymes into products that emit light, and other reagents used in performance of the assay method.

In another aspect, the invention provides a method for determining if a physical, chemical, dietary, or pharmacological treatment of interest stimulates or inhibits cell proliferation in a tissue of a mammal, comprising treating the mammal with the treatment of interest, administering an amount of a 5-halo-2′-deoxyuridine (HdU) to the mammal sufficient to effect incorporation of HdU into newly synthesized DNA of proliferating cells in the mammal, removing a tissue sample from the mammal, extracting genomic DNA from cells of the tissue sample, contacting the genomic DNA with monoclonal anti-HdU antibodies under conditions in which the antibodies bind to HdU-containing DNA, and an enzyme that catalyzes conversion of a substrate to a light-producing product is attached to the anti-HdU antibodies that are bound to the HdU-containing DNA, contacting the anti-HdU antibodies that are bound to the HdU-containing DNA with a solution containing a substrate compound that is converted to a light-emitting product by the enzyme, at a sufficient substrate concentration and under conditions such that a chemiluminescent signal is emitted, measuring the intensity of the chemiluminescence to detect and quantify the amount of HdU incorporated into the DNA, and comparing the amount of HdU incorporated into the DNA with the amount of HdU incorporated into DNA extracted from the same type of tissue of a control mammal that has not received the treatment of interest, determined in like manner, to determine if the treatment stimulates or inhibits cell proliferation in the tissue. For example, the treatment of interest may comprise administering a compound to the mammal, in which case the method would comprise comparing the amount of HdU incorporated into the DNA with the amount of HdU incorporated into the DNA extracted from the same tissue of a control mammal to which the compound has not been administered, to determine if the compound stimulates or inhibits cell proliferation in the tissue. The control mammal can be the same individual as the assay subject mammal, or a different individual.

In the method for determining if a physical, chemical, dietary, or pharmacological treatment of interest stimulates or inhibits cell proliferation in a tissue of a mammal, the HdU may be administered by any route, including but not limited to intraperitoneal, intravenous, intramuscular, and subcutaneous routes. The method may be performed by administering a dosage of from 25 to 1500 mg/kg of the HdU to the mammal in one day. The HdU may be administered once or several times in one day, and it may be administered on one or multiple days. BrdU can be used as the HdU. For example, the method can be performed by administering a dosage of from 25 to 1500 mg/kg of BrdU to the mammal in one day. The tissue sample on which the assay is performed may be any tissue of the mammal; e.g., blood, bone marrow, pancreas, liver, kidney, intestines, thymus, heart, striated muscle, spinal cord, or brain. For example, the method may comprise using a tissue sample that includes part or all of the hippocampus.

Additional details and aspects of the invention are described below.

### BRIEF DESCRIPTION OF THE DRAWINGS

FIG. 1 schematically depicts an anti-HdU antibody attached to the inner surface of an assay well that is bound to HdU-containing DNA. An enzyme linked anti-HdU antibody is also bound to the HdU-containing DNA, and the curved arrow represents catalysis by the enzyme to convert the substrate into a light-emitting substrate.

FIG. 2 is a graph showing calorimetric data (absorbance at 450 nm) produced by a 1-step BrdU calorimetric ELISA for samples containing from 1 to >1000 ng BrdU(+) DNA in a total of 5 μg DNA.

FIG. 3 is a graph showing calorimetric data (absorbance at 450 nm) produced by a 2-step BrdU calorimetric ELISA for samples containing from 0 to 50 ng BrdU(+) DNA in a total of 5 μg DNA.

FIG. 4 is a graph showing raw chemiluminescence data produced by a 1-step BrdU chemiluminescence ELISA for samples containing from 0 to 100 ng BrdU(+) DNA in a total of 5 μg DNA.

FIG. 5 is a graph showing the chemiluminescence data of FIG. 3 after subtraction of background chemiluminescence, for samples containing from 0 to 100 ng BrdU(+) DNA in a total of 5 μg DNA.

FIG. 6 is a graph showing raw chemiluminescence data produced by a 1-step BrdU chemiluminescence ELISA for samples containing from 0 to 75 ng BrdU(+) DNA in a total of 5 μg DNA.

FIG. 7 is a graph showing the chemiluminescence data of FIG. 5 after subtraction of background chemiluminescence, for samples containing from 0 to 75 ng BrdU(+) DNA in a total of 5 μg DNA.

FIG. 8 is a graph comparing the lower limits of sensitivity for BrdU(+) DNA of a 1-step BrdU calorimetric ELISA, a 2-step BrdU calorimetric ELISA, and a 1-step chemiluminescence BrdU ELISA.

FIGS. 9A-9C schematically depicts sections of the brain through the hippocampus (FIG. 9A), and provides a graphic depiction (FIG. 9C) of proliferating cells stained within the subgranular zone of the dentate gyrus of the hippocampus (FIG. 9B).

FIG. 10 is a graph showing chemiluminescence data for BrdU(+) DNA isolated from hippocampus of rats that received single, daily ip. injections of 100 mg/kg BrdU on one, four, or seven consecutive days.

FIG. 11 is a graph showing chemiluminescence data for BrdU(+) DNA isolated from hippocampus of rats that received single, daily ip. injections of 100 mg/kg BrdU on one or four consecutive days, and from hippocampus of rats that received four ip. injections of 225 mg/kg BrdU on a single day, at 2-hour intervals.

FIG. 12 is a graph showing chemiluminescence data for BrdU(+) DNA isolated from hippocampus punch samples and from whole, dissected hippocampus.

FIG. 13 is a graph showing chemiluminescence data for BrdU(+) DNA extracted from whole hippocampus of mammals that received on one day four ip. injections of 25, 50, or 100 mg/kg BrdU, or a single ip. injection of 200 mg/kg BrdU (experiment 1).

FIG. 14 is a graph showing chemiluminescence data for BrdU(+) DNA extracted from whole hippocampus of mammals that received on one day four ip. injections of 25, 50, or 100 mg/kg BrdU (experiment 1, also shown in FIG. 13), or four ip. injections of 100, 150, or 225 mg/kg BrdU (experiment 2).

FIG. 15 is a graph showing chemiluminescence data for assay wells that received 5 μg or 10 μg of genomic DNA extracted from whole hippocampus of mammals that received on one day four ip. injections of 100, 150, or 225 mg/kg BrdU.

FIG. 16 is a graph showing chemiluminescence data for BrdU(+) DNA extracted from whole hippocampus of mammals that received four ip. injections of 100 or 150 mg/kg BrdU on one day following ECS treatment.

FIG. 17 is a graph showing chemiluminescence data for BrdU(+) DNA extracted from whole hippocampus of mammals that received four ip. injections of 100 or 150 mg/kg BrdU on one day following ECS treatment.

### DETAILED DESCRIPTION OF THE INVENTION

The present invention uses a chemiluminescence HdU ELISA in a rapid, sensitive method for detecting and quantifying cell proliferation in a mammal. The method of the invention comprises the following steps (a)-(f):
(a) a subject mammal is administered an amount of a 5-halo-2′-deoxyuridine (HdU) that is sufficient to effect incorporation of HdU into newly synthesized DNA of proliferating cells in the mammal.
(b) a tissue sample is removed from the subject mammal;
(c) genomic DNA is extracted from cells of the tissue sample;
(d) the genomic DNA of step (c) is contacted with monoclonal anti-HdU antibodies under conditions in which the antibodies bind to HdU-containing DNA, and wherein an enzyme that catalyzes conversion of a substrate to a light-producing product is attached to the anti-HdU antibodies that are bound to the HdU-containing DNA;
(e) the anti-HdU antibodies that are bound to the HdU-containing DNA are contacted with a solution containing a substrate compound that is converted to a light-emitting product by the enzyme of step (d), at a sufficient substrate concentration and under conditions such that a chemiluminescent signal is emitted; and
(f) the intensity of the chemiluminescence produced in step (e) is determined.

The method of the invention can be performed to detect and quantify proliferation of cells in any tissue of any mammal. Accordingly, mammals in which cell proliferation can be quantified by the invention include mouse, rat, cat, dog, pig, non-human primate, and human. The mammalian subject of the method may be of any age or developmental stage; e.g., it can be an embryonic, fetal, infantile, young, juvenile, adolescent, or adult. Adult mammals that can be used for the invention include mammals that are young adults, middle-aged, or old-aged, relative to the average life-span of the species.

A mammal that is a subject of the method can be treated with a physical, chemical, dietary, or pharmacological treatment that stimulates or inhibits cell proliferation in the tissue of interest, or has the potential to do so, prior to performing the method of the invention. Such treatment includes any physical treatment that impinges upon the senses of the mammal, including sights, sounds, smells, and tactile stimulation. For example, a subject mammal may engage in voluntary exercise, or may be administered an antidepressant medication or subjected to electroconvulsive shock (ECS), any of which may stimulate cell division in the hippocampus. Alternatively, the mammal may be subjected to stress, e.g., by mild immobilization, or may be administered an opiate, which treatments inhibit cell division in the hippocampus. Methods for providing such treatments are well-known. Additional treatments that may stimulate or inhibit cell proliferation in a tissue of interest include administration of growth factors or cytokines, and transplantation of autologous or heterologous precursor cells or stem cells into a tissue of interest. The invention includes but is not limited to treatments that potentially stimulate or inhibit cell proliferation in a tissue of interest such as those identified above. Any treatment that can potentially stimulate or inhibit cell proliferation in a tissue of interest may be provided to the subject mammal prior to performing, and in conjunction with, the assay method of the invention. For example, the invention can be used to test compounds of unknown activity to determine if they are capable of stimulating or inhibiting cell proliferation in tissues of mammals. The assay method may be used to test compounds to determine if they stimulate or inhibit cell proliferation in the hippocampus.

The method of the invention can be performed with any tissue of a subject mammal in which there are cells capable of proliferating. For example, the assay can be performed with a tissue sample that comprises but is not limited to one or more of blood, bone marrow, pancreas, liver, kidney, intestines, thymus, heart, striated muscle, spinal cord, and brain. Examples of brain tissues that can be used in the assay method include the hippocampus and the subventricular zone adjacent to a lateral ventricle. In one aspect of the invention, the cells in the tissue of interest that are capable of proliferating are autologous or heterologous cells, e.g., precursor or stem cells, that are introduced into the tissue by known transplantation methods.

HdU that are suitable for use in the assay method of the invention are chemical analogs of thymidine that can cross the blood/brain barrier and are readily taken up by cells in vivo and incorporated into their DNA. Examples of such HdU that can be used for the invention include 5-bromo-2′-deoxyuridine (BrdU), 5-iodo-2′-deoxyuridine (IdU), and 5′-chloro-2′-deoxyuridine (CldU), which are readily available commercially available (e.g., from Sigma, St. Louis, Mo.).

The selected HdU is administered to a subject mammal at a sufficient concentration and in an amount and manner such that HdU nucleosides are incorporated into newly synthesized DNA of proliferating cells in the mammal. Methods for administering HdU nucleosides to a mammal to successfully effect incorporation of HdU into new DNA in proliferating cells in the mammal are known. A method for administering BrdU to a mammal to effect BrdU incorporation into DNA of cells proliferating in hippocampus and other tissues is described in the working example disclosed below. The HdU can be administered once or several times in one day, and it may be administered on one or multiple days. For example, a dosage of from 25 to 1500 mg/kg of the HdU can be administered to the mammal on one day, either as a single injection or in several injections administered at intervals. Additional dosages may be administered on following days. A suitable protocol comprises administering several consecutive intraperitoneal (ip.) injections of HdU in saline at a concentration of from about 25 to about 200 mg/kg, at 1-3 hour intervals. For example, detectable amounts of BrdU are incorporated into DNA of cells proliferating in a tissue of a mammal following 4 consecutive ip. injections of BrdU in PBS (15 mg/ml) at a dosage of 100 mg/kg in a 6.7 ml/kg injection volume, given at 2 hour intervals. Other protocols for administering HdU nucleosides to a mammal that successfully effect incorporation of HdU into the DNA of cells proliferating in the mammal's tissues can be identified through routine testing, using the assay method of the invention, with an HdU administration protocol of known effectiveness as a control.

The amount of HdU incorporated into the DNA of proliferating cells depends in part on the amount of time that the cells are dividing in the presence of the HdU. For example, the DNA of cells grown in vitro for 24 hours in the presence of BrdU generally contains significantly more BrdU than the DNA of cells grown in vitro for only 4 hours in the same concentration of BrdU. The amount of HdU incorporated into the DNA of proliferating cells also depends on the concentration of BrdU to which the cells are exposed. Using the assay of the present invention to measure cell proliferation in hippocampus tissue of rats in vivo, it can be shown that BrdU incorporation into genomic DNA increases in a dose-dependent manner following administration of BrdU at dosages of from 25 to 100 mg/kg per injection as described above (4 ip. injections at 2-hour intervals); however, at BrdU doses higher than 100 mg/kg, there is little additional increase in BrdU incorporation into DNA. See Example 6.

Depending on the number of cells that are proliferating in the tissue(s) of interest and the dosage of HdU that is administered, HdU incorporation into DNA by cells in vivo can be detected and quantified by the assay method of the invention in tissue removed from the mammal as early as one to four hours following HdU administration. HdU incorporation into DNA by cells in vivo can also be detected and quantified by the assay method in tissues that are removed from the mammal four to 24 hours, or one to seven days, following HdU administration, as well as in tissues that are removed from one to six weeks or more following HdU administration. The dependence of the amount of HdU that is incorporated into DNA by cells in vivo on the time period between the administration of HdU to the subject mammal and the removal of the tissue of interest can be determined by routine experimentation, using the assay method of the invention.

Following administration of HdU to a subject mammal, a tissue sample comprising cells that have incorporated HdU into their genomic DNA is removed from the subject mammal using a known method; e.g., by surgical dissection and excision, or by surgical or needle biopsy. Tissue samples may be frozen and stored (e.g., at −80° C.) until DNA is extracted.

Genomic DNA is extracted from cells of the tissue sample using known methods. For example, genomic DNA can be extracted rapidly and efficiently using a commercially available kit for extracting DNA from mammalian tissues and cells, such as the DNEASY® Tissue Kit (Qiagen, Valencia, Calif., cat. no. 69504), which provides a spin column that contains a silica gel membrane that selectively binds DNA and allows contaminants to pass through. In accord with the kit protocol, a tissue sample is lysed and cellular proteins are degraded by incubation in buffered solution containing proteinase K, the lysate is loaded onto the spin column and spun, DNA bound to the membrane is washed, and purified DNA is eluted in water or low salt buffer. Other known methods for extracting genomic DNA from tissue that provide a preparation of genomic DNA that is relatively free of proteins and other non-nucleic acid molecules can also be used effectively for the assay method of the invention.

After genomic DNA is extracted, it is contacted with monoclonal anti-HdU antibodies under conditions in which the antibodies bind to the HdU-containing DNA. Monoclonal antibodies that bind specifically to 5-bromo-2′-deoxyuridine (BrdU), 5-iodo-2′-deoxyuridine (IdU), or 5′-chloro-2′-deoxyuridine (CldU), that are suitable for use in the assay method of the invention are readily available commercially. The genomic HdU-containing DNA generally must be at least partially denatured for some anti-HdU antibodies to bind to HdU nucleosides that are incorporated into the DNA. Methods for denaturing the DNA, e.g., by heating, prior to contacting with such anti-HdU antibodies are well-known. The genomic DNA obtained using known methods for extracting genomic DNA from mammal tissue is often partially denatured, so that an additional step to denature the DNA prior to contacting with anti-HdU antibodies is commonly not required for effective operation of the invention.

The method of the invention utilizes a chemiluminescence enzyme-linked immunosorbent assay (ELISA)—it uses an enzyme linked to anti-HdU antibodies that bind to the HdU-containing DNA in the assay sample to generate a chemiluminescent signal, and measures the intensity of the chemiluminescent signal to detect and quantify the extracted HdU-containing DNA. By comparing the chemiluminescent signal obtained for HdU-containing DNA from tissue labeled with HdU in vivo with results obtained by performing the assay with known amounts of HdU-containing DNA from cells labeled in vitro, one can estimate the amount of HdU-containing DNA present in a tissue sample from a subject mammal, from the intensity of the chemiluminescent signal that is measured by the assay.

Anti-HdU antibodies useful in the disclosed methods include intact monoclonal antibodies, polyclonal antibodies, multispecific antibodies (e.g. bispecific antibodies) formed from at least two intact antibodies, humanized antibodies, primatized antibodies, domain-deleted antibodies, and antibody fragments so long as they exhibit the desired biochemical activity.

Antibody fragments that specifically bind HdU may also be used, for example, a portion of an intact antibody comprising the variable region or antigen binding region thereof. Examples of antibody fragments include Fab, Fab′, F(ab′)₂, and Fv fragments; diabodies; linear antibodies; single chain antibody molecules; and multispecific antibodies formed from antibody fragments. Domain-deleted antibodies comprising immunoglobulins of which at least part of one or more constant regions have been altered or deleted are also considered antibody fragments as the term is used herein.

As used throughout the entire application, the terms “a” and “an” are used in the sense that they mean “at least one,” “at least a first,” “one or more,” or “a plurality” of the referenced components or steps, except in instances wherein an upper limit is thereafter specifically stated. Accordingly, reference to an “antibody” as used herein means “at least a first antibody.”

The disclosed methods can be performed using anti-HdU antibodies that are covalently linked to the enzyme that produces the chemiluminescent product, in a “1-step” protocol. Alternatively, the enzyme can be covalently linked to a secondary agent such as a secondary antibody, and its linkage to the anti-HdU antibodies can be effected by binding the enzyme-linked secondary agent to the (primary) anti-HdU antibodies that are bound to the HdU-containing DNA, in a “2-step” protocol. Various methods for effecting attachment of the enzyme to the primary antibody are well known. For example, the primary antibody can be covalently linked to a ligand (e.g., biotin), and the enzyme can be covalently linked to a receptor of the ligand (e.g., avidin). The binding of avidin to its ligand biotin would then effect attachment of the enzyme to the primary antibody. In a variation of the 2-step protocol, both the primary anti-HdU antibodies and the secondary agent are linked to enzyme molecules that produce the chemiluminescent product. In another variation, the secondary antibody is biotinylated, and after the secondary antibody is bound to the primary antibody, the enzyme is attached to the secondary antibody through the binding of avidin to biotin. The scientific and technical literature describes other ways for effecting attachment of the enzyme to the primary antibody for carrying out chemiluminescence HdU ELISA in practicing the invention.

The assay method of the invention requires at least one step of washing the HdU-containing DNA to remove unbound and non-specifically bound reaction components (i.e., the anti-HdU antibodies and/or the secondary agent) before adding reaction substrate and other reagents required to produce a chemiluminescent signal. Washing the HdU-containing DNA is facilitated by first allowing it to bind to an insoluble support material (solid phase) that is capable of binding to the DNA. Insoluble support materials that bind DNA and permit washing and analysis of the bound DNA which are suitable for use in the assay method of the invention are well known. Such insoluble support materials include any solid support that is capable of binding DNA, or that is capable of being chemically modified to bind DNA. Known support materials suitable for the invention include glass, gold, hydroxyapatite, polystyrene, polypropylene, polyethylene, dextran, nylon, amylases, natural and modified celluloses (e.g., nitrocellulose), polyacrylamides, agaroses, and silica-magnetite. The support material may have virtually any possible structural configuration so long as DNA bound to the support material is also capable of binding to an antibody. Thus, the support configuration may be spherical (e.g., a bead), cylindrical or curved, as in a test tube or assay well, or the external surface of a rod. Alternatively, the surface may be flat, e.g., a sheet, membrane, test strip, etc., or the bottom of an assay well. Alternatively, the support surface may be a polymeric matrix, e.g., a chromatographic resin. Those skilled in the art will be familiar with these and other suitable support materials carriers for binding DNA, or will be able to ascertain the same by use of routine experimentation. The HdU-containing DNA can remain bound to the insoluble support material during subsequent steps of the assay, or it can be released from the insoluble support after the wash steps are completed.

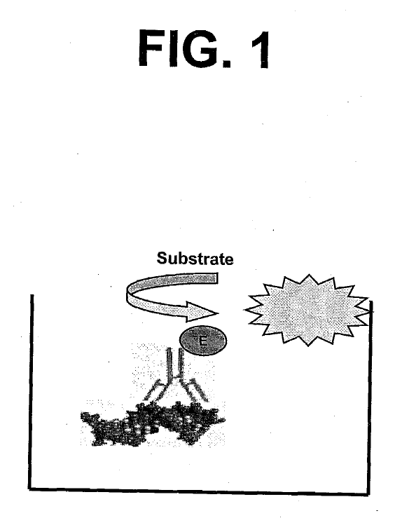

The surface of the support material can be chemically modified so that it is capable of binding to DNA, e.g., by coating the surface with a chemical agent that binds to DNA. For example, an inner surface of a container or well in which the assay is performed can be coated with a DNA-binding protein. The DNA-binding protein can be any protein that binds to DNA with relatively high affinity and specificity; e.g., an antibody that binds specifically to DNA. For example, the inner surface of an assay well can be coated with antibodies (monoclonal or polyclonal) that bind specifically to HdU nucleosides incorporated into DNA. Upon adding the genomic DNA to the assay well, the HdU-containing DNA molecules present in the genomic DNA bind to the anti-HdU antibodies coating the assay well surface, and the DNA molecules that do not contain HdU are removed by washes. In the 1-step chemiluminescence HdU ELISA of the present invention, enzyme-linked anti-HdU antibodies are added to the assay well and allowed to bind to the HdU-containing DNA, and unbound anti-HdU antibodies are removed by washing. The DNA in effect is “sandwiched” between the anti-HdU antibodies bound to the solid support and the enzyme-linked anti-HdU antibodies. Upon addition of the substrate of the chemiluminescence reaction, the enzyme that is attached to the anti-HdU antibodies catalyzes conversion of the substrate into a light-emitting product, as shown in FIG. 1. The amount of light that is produced is quantitatively dependent on the number of enzyme molecules present in the assay well, which in turn is a function of the number of HdU nucleosides present in the DNA that is bound in the well. The enzyme attached to the anti-HdU antibodies can be any enzyme that catalyzes conversion of a substrate to a light-producing product. Examples of enzymes that can be used for the assay method include peroxidase (POD), alkaline phosphatase, β-galactosidase, acid phosphatase, and β-glucuronidase. Such enzymes and their light-emitting substrates (and reagents facilitating their use) are commercially available. Suitable substrates include diacylhydrazides such as luminol. In the presence of hydrogen peroxide, POD catalyzes the oxidation of a diacylhydrazide such as luminol to form a reaction product in an excited state, which decays to a ground state by emitting light. Reliable methods are known that can be used to attach anti-HdU antibodies to an enzyme that catalyzes production of a light-emitting product for use in practicing the invention. In addition, enzyme-linked anti-HdU antibodies for chemiluminescence HdU ELISA that are suitable for the present invention are commercially available. An example is anti-BrdU peroxide (POD)-conjugated Fab antibody fragment (Roche Applied Science, Indianapolis, Ind., cat. no. 1585860), which catalyzes the conversion of BM Chemiluminescence ELISA Substrate (POD) (Roche Applied Science, cat. no: 1 582 950) into a light emitting substrate. BM Chemiluminescence ELISA Substrate (POD) is a buffered solution containing luminol, 4-iodophenol, and a stabilized form of hydrogen peroxide (H₂O₂). In the presence of hydrogen peroxide H₂O₂, horseradish peroxidase (POD) catalyzes the oxidation of luminol to form a reaction product in an excited state that emits light when it decays to the ground state. Strong enhancement of the light emission is achieved by the agent 4-iodophenol in the reaction solution, which acts as a radical transmitter between the formed oxygen radical and luminol. The light-emitting reaction reaches steady state within 2-3 minutes and can be detected and quantified by a typical luminometer with photomultiplier technology, such as an Autolumat LB 953 tube chemiluminescence analyzer (EG&G Berthold, Oak Ridge, Tenn.). Luminometers designed to detect chemiluminescence in the wells of a microplate, such as Microplate Luminometer LB96 (EG&G Berthold, Bad Wildbad, Germany) are useful for practicing the invention and are readily available. Chemiluminescence is quantified in relative light units/second (rlu/sec). The number of rlu/sec detected by the method of the invention directly correlates with the amount of enzyme-linked anti-HdU antibodies bound to the DNA, and therefore to the amount of HdU-containing DNA, which is proportional to the number of proliferating cells in the sample of interest.

The invention provides a method for determining if a physical, chemical, dietary, or pharmacological treatment of interest stimulates or inhibits cell proliferation in a tissue of a mammal, comprising:
(a) treating the mammal with the treatment of interest;
(b) administering an amount of a 5-halo-2′-deoxyuridine (HdU) to the mammal sufficient to effect incorporation of HdU into newly synthesized DNA of proliferating cells in the mammal;
(c) removing a tissue sample from the mammal;
(d) extracting genomic DNA from cells of the tissue sample;
(e) contacting the genomic DNA of step (d) with monoclonal anti-HdU antibodies under conditions in which the antibodies bind to HdU-containing DNA, and an enzyme that catalyzes conversion of a substrate to a light-producing product is attached to the anti-HdU antibodies that are bound to the HdU-containing DNA;
(f) contacting the anti-HdU antibodies that are bound to the HdU-containing DNA with a solution containing a substrate compound that is converted to a light-emitting product by the enzyme of step (e), at a sufficient substrate concentration and under conditions such that a chemiluminescent signal is emitted;
(g) determining the intensity of the chemiluminescence produced in step (f) to detect and quantify the amount of HdU incorporated into the DNA; and
(h) comparing the amount of HdU incorporated into the DNA with the amount of HdU incorporated into DNA extracted from the same type of tissue of a control mammal that has not received the treatment of interest, as determined by steps (b)-(g), to determine if the treatment stimulates or inhibits cell proliferation in the tissue.

As described above, the mammal can be treated with any physical, chemical, dietary, or pharmacological treatment that stimulates or inhibits cell proliferation in a tissue of interest, or has the potential to do so. Such treatment includes any physical treatment that impinges upon the senses of the mammal, including sights, sounds, smells, and tactile stimulation. Examples include exercise, administration of a drug, or a directed physical treatment such as electroconvulsive shock (ECS), which have been shown to stimulate cell division in the hippocampus. Alternatively, the mammal may be subjected to a treatments that inhibit cell division in a tissue; for example, stress, e.g., by mild immobilization, and administration of an opiate have been shown to inhibit cell division in the hippocampus. Methods for providing these and many other such treatments are well-known. Additional treatments that may stimulate or inhibit cell proliferation in a tissue of interest include administration of growth factors or cytokines, and transplantation of autologous or heterologous precursor cells or stem cells into a tissue of interest. The invention includes but is not limited to treatments that potentially stimulate or inhibit cell proliferation in a tissue of interest such as those identified above. Any treatment that can potentially stimulate or inhibit cell proliferation in a tissue of interest may be provided to the subject mammal in performing the method of the invention.

When the treatment of interest of step (a) comprises administering a compound to the mammal, step (h) would comprise comparing the amount of HdU incorporated into the DNA with the amount of HdU incorporated into the DNA extracted from the same tissue of a control mammal to which the compound has not been administered as determined by steps (b)-(g) to determine if the compound stimulates or inhibits cell proliferation in the tissue. As described above for the detection method, the HdU can be administered by any route, including but not limited to intraperitoneal, intravenous, intramuscular, and subcutaneous. For example, Step (b) of the method can be performed by administering a dosage of from 25 to 1500 mg/kg of the HdU to the mammal in one day. The HdU can be administered once or several times in one day, and it may be administered on one or multiple days. BrdU can be used as the HdU. For example, the method can be performed by administering a dosage of from 25 to 1500 mg/kg of BrdU to the mammal in one day. The tissue sample removed in step (c) may be any tissue of the mammal; e.g., blood, bone marrow, pancreas, liver, kidney, intestines, thymus, heart, striated muscle, spinal cord, or brain. In a useful form of the invention, the method comprises using a tissue sample of step (c) that includes part or all of the hippocampus.

### EXAMPLES

### Example 1

### Sensitivity of a One-Step Calorimetric BrdU ELISA

BrdU-Containing DNA from Cultured Cells Chinese Hamster Ovary Cells (CHO-KI cell line) were grown in Dulbecco's Modified Eagle's Medium (DMEM) medium with 10% fetal bovine serum (FBS) in T 175 flasks and incubated with 100 μM BrdU for 48 hours. The cells were harvested and BrdU-containing genomic DNA was extracted and purified using a Cell Culture DNA Maxi Kit (Qiagen, cat. no. 13362), which provides a mini-column containing an anion-exchange resin that selectively binds DNA and allow contaminants to pass through. Following the maxi kit protocol, the cells were lysed in buffered proteinase K solution, the lysate was loaded onto the mini-column, DNA bound to the column resin was washed, and purified DNA was eluted with elution buffer.

### 1-Step Calorimetric BrdU ELISA

A 1-step calorimetric BrdU ELISA was performed in 96 well NUNC® Maxisorb microtiter plates. The plates were coated with 100 μl/well of 1:100 dilution of monoclonal anti-BrdU antibodies (BD Biosciences, San Jose, Calif., cat. no. 347580) in coating buffer (0.05M carbonate buffer, pH 9.6) and incubated overnight at 4° C. on a shaker. This was followed by blocking with 1% bovine serum albumin (BSA) in phosphate buffered saline (PBS) for 60 minutes, and 5 washes with TBST buffer (20 mM Tris pH 7.6, 150 mM NaCl, 0.05% Tween 20). A selected amount of BrdU(+) DNA mixed with unlabeled DNA to give a total of 5 μg DNA in 100 μl buffer was added to each well and incubated. The wells were then washed 5 times with TBST buffer, followed by incubation with anti-BrdU peroxide (POD)-conjugated Fab antibody fragment (Roche Applied Science, Indianapolis, Ind., cat. no. 1585860). The wells were washed 5 time again with TBST buffer, and POD activity was determined by adding 100 μl TMB (Roche Applied Science, Indianapolis, Ind.) substrate per well for 15 minutes, stopping the reaction by adding 25 μl 1 M H₂SO₄, and determining absorbance at 450 nm.

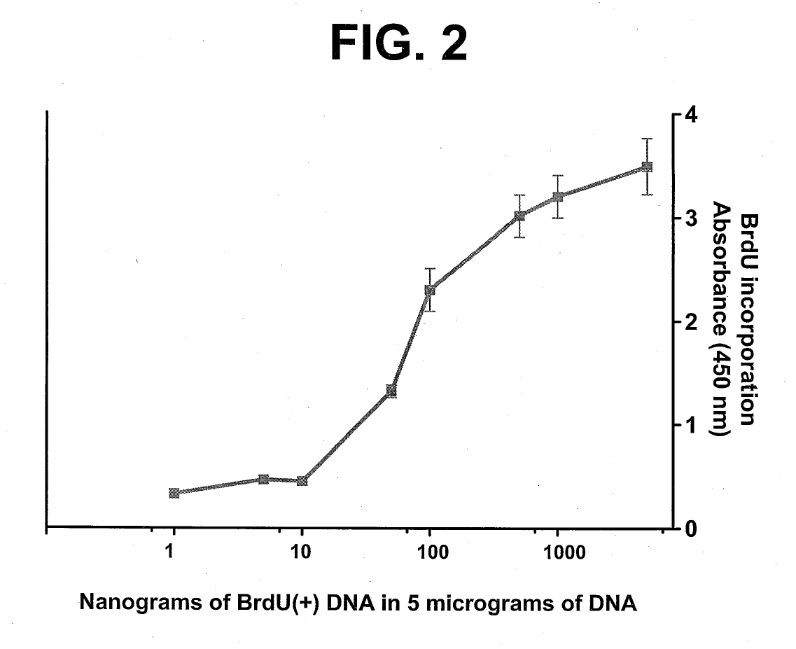

As shown in FIG. 2, the assay detects BrdU(+) DNA isolated from cultured cells in a concentration-dependent manner. From FIG. 2, it can also be seen that the lower limit of detection of the single step BrdU calorimetric ELISA is about 50 ng BrdU(+) DNA in a total of 5 μg DNA. Assuming that 1 ng of genomic DNA corresponds to about 100 cells, the single step BrdU calorimetric ELISA can detect BrdU(+) DNA of about 5000 BrdU(+) cells in a total of about 500,000 cells.

### Example 2

### Sensitivity of a Two-Step Calorimetric BrdU ELISA

BrdU-Containing DNA from Cultured Cells

CHO-KI cells were grown in medium containing 100 μM BrdU for 48 hours, the cells were harvested, and BrdU-containing genomic DNA was extracted and purified as described above.

### 2-Step Calorimetric BrdU ELISA

A two-step calorimetric BrdU ELISA was performed. Sheep polyclonal anti-BrdU antibodies (Novus Biologicals, Littleton, Colo.) were used as the coating antibodies. Coating antibody dilutions of 1:100, 1:1000, 1:2000, and 1:5000 were tested, and coating antibody dilution of 1:2000 was selected as giving a suitable signal/background ratio. The assay was performed using NUNC® Maxisorb microtiter assay plates. After the wells were coated, blocking using 2% BSA, 5% BSA, and SUPERBLOCK® (Pierce) was tested, and SUPERBLOCK® (Pierce) was selected as giving suitable results. A selected amount of BrdU(+) mixed with unlabeled DNA to give a total of 5 μg DNA in 100 μl buffer was added to each well and incubated for 120 minutes. The wells were then washed 5 times with TBST buffer, followed by incubation with the primary (1°) antibody. Monoclonal murine anti-BrdU antibody Fab fragment linked to peroxidase (POD) (Roche Applied Science, Indianapolis, Ind., cat. no. 1585860) was used as the 1° antibody. After testing PBS, TBS, PBS+1% BSA, and PBS+3% BSA as 1° antibody dilution buffers, PBS+1% BSA was selected as an antibody dilution buffer giving suitable results. 1° antibody dilutions of 1:100, 1:200, and 1:500 were tested, and 1° antibody dilution of 1:100 was selected as giving suitable signal/background ratio. Following incubation of the 1° antibody with the BrdU(+) DNA in the well, wash solutions (TBST, PBS, and TBST with final 2 washes in PBS) were tested (5 washes, 5 minutes per wash), and TBST with final 2 washes in PBS was chosen as giving suitable results.

A Fab-specific anti-mouse IgG antibody linked to POD was selected as the secondary (20) antibody. 20 antibody dilutions of 1:1000, 1:10,000, 1:15,000, 1:20,000 were tested and 1:10,000 was selected as giving suitable results. After testing PBS, TBS, PBS+1% BSA, and PBS+3% BSA, PBS+1% BSA was chosen as a 20 antibody dilution buffer that gives a suitable signal/background ratio. Following incubation of the antibody with the 1° antibody bound to BrdU(+) DNA in the assay wells, the wells were washed 5 times for 5 minutes per wash using TBST with final 2 washes in PBS, as described above. POD activity was determined by adding 100 μl TMB substrate to each and incubating for 15 minutes, stopping the reaction by adding 25 μl 1 M H₂SO₄, and determining absorbance at 450 nm, as described for the 1-step calorimetric ELISA.

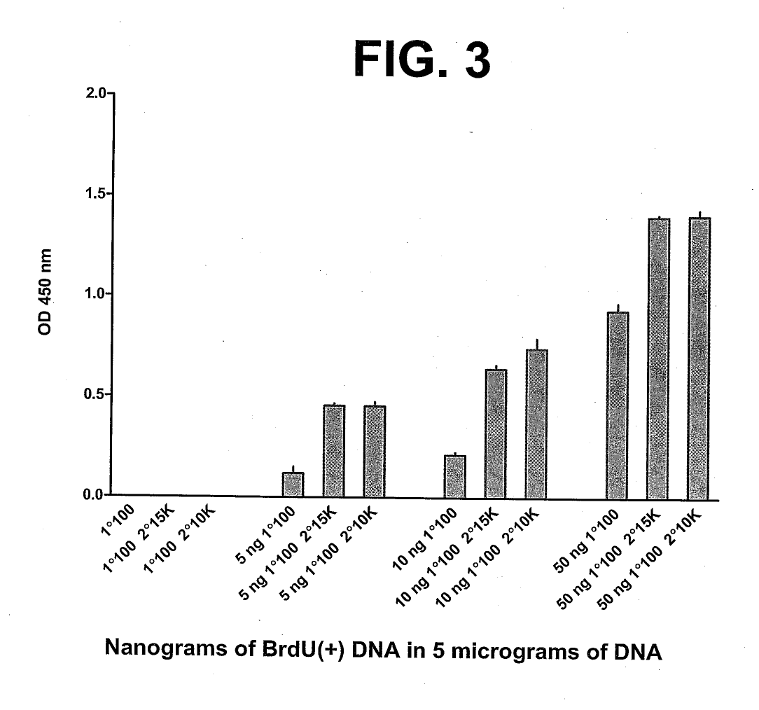

Results of the 2 step BrdU calorimetric ELISA for assay wells containing 0, 5, 10, and 50 ng BrdU(+) DNA in a total of 5 μg DNA, for 20 antibody dilutions of 1:100, 1:10,000, and 1:15,000, are shown in FIG. 3. The plotted data are values of absorbance at 450 nm after subtracting background values of <0.30 OD 450 nm. As shown in FIG. 3, 5 ng of BrdU(+) DNA in a total of 5 μg DNA is at the lower limit of detection of the 2-step BrdU calorimetric ELISA. Assuming that 1 ng of DNA corresponds to about 100 cells, the 2-step BrdU calorimetric ELISA can detect BrdU(+) DNA of about 500 BrdU(+) cells in a total of about 500,000 cells.

### Example 3

### Sensitivity of a Chemiluminescence BrdU ELISA

BrdU-Containing DNA from Cultured Cells

CHO-KI cells were grown in medium containing 100 μM BrdU for 48 hours, the cells were harvested, and BrdU-containing genomic DNA was extracted and purified as described above.

### Detection of BrdU(+) DNA by Chemiluminescence BrdU ELISA

Costar high binding 96 well flat bottom polystyrene plates (Corning Inc., Acton, Mass., cat. no. 3601) were selected as microtiter plates for the assay, and sheep polyclonal anti-BrdU antibodies (Novus Biologicals, Littleton, Colo.) were used as the coating antibodies. After testing dilutions of 1:100 and 1:2000, coating antibody dilution of 1:2000 was selected as giving suitable signal/background. PBS, PBS+1% BSA, PBS+3% BSA, and Roche antibody dilution buffer were tested, and Roche antibody dilution buffer was selected as an antibody dilution buffer that gives suitable results. Wash conditions selected were the same as those described above; i.e., 5 washes (5 minutes per wash) using TBST with final 2 washes in PBS. A selected amount of BrdU(+) mixed with unlabeled DNA to give a total of 5 μg DNA in 100 μl buffer was added to each well and incubated. The wells were then washed 5 times using TBST with final 2 washes in PBS.

Monoclonal murine anti-BrdU antibody Fab fragment linked to peroxidase (POD) (Roche Applied Science, Indianapolis, Ind., cat. no. 1585860) was used as the 1° antibody. After testing PBS, TBS, PBS+1% BSA, and PBS+3% BSA as 1° antibody dilution buffers, PBS+1% BSA and PBS+3% BSA were found to give suitable results. 1° antibody dilutions of 1:100, 1:200, and 1:500 were tested, and 1° antibody dilution of 1:100 was selected as giving suitable signal/background ratio. The 1° antibody at a dilution of 1:100 was added to each well and incubated overnight at 4° C. Following incubation with the 1° antibody, the wells were washed 5 times, 5 minutes per wash, using TBST and final 2 washes in PBS. The reaction producing chemiluminescence was initiated by addition of 100 μl of BM Chemiluminescence ELISA Substrate (POD) (Roche Applied Science, cat. no: 1 582 950) according to the manufacturer's instructions, and chemiluminescence was determined using a standard luminometer.

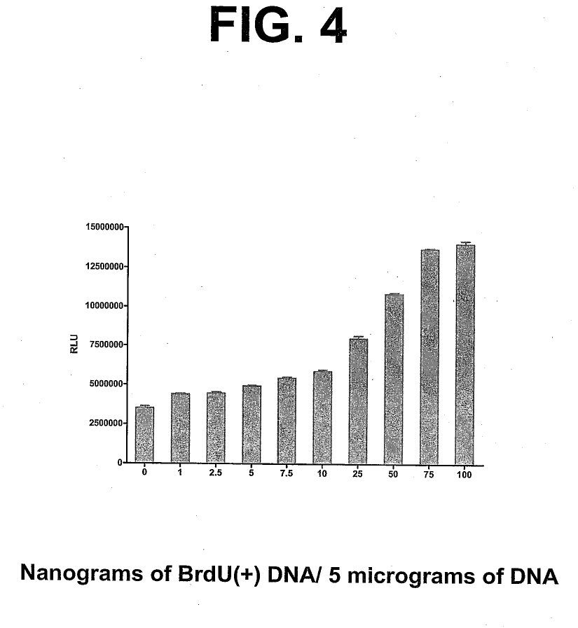

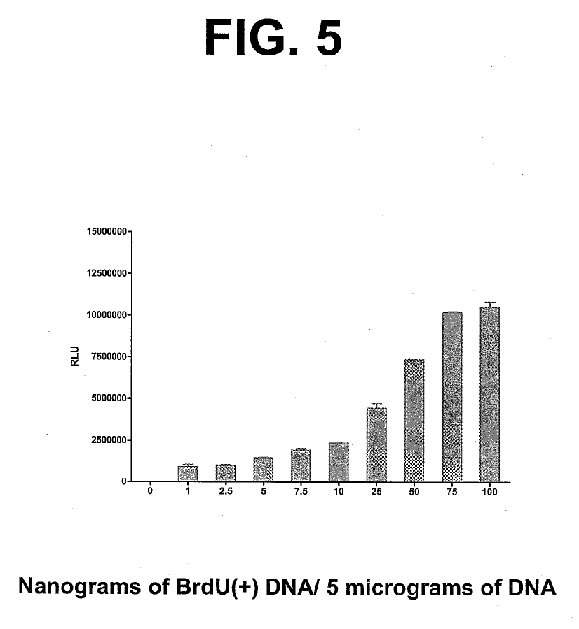

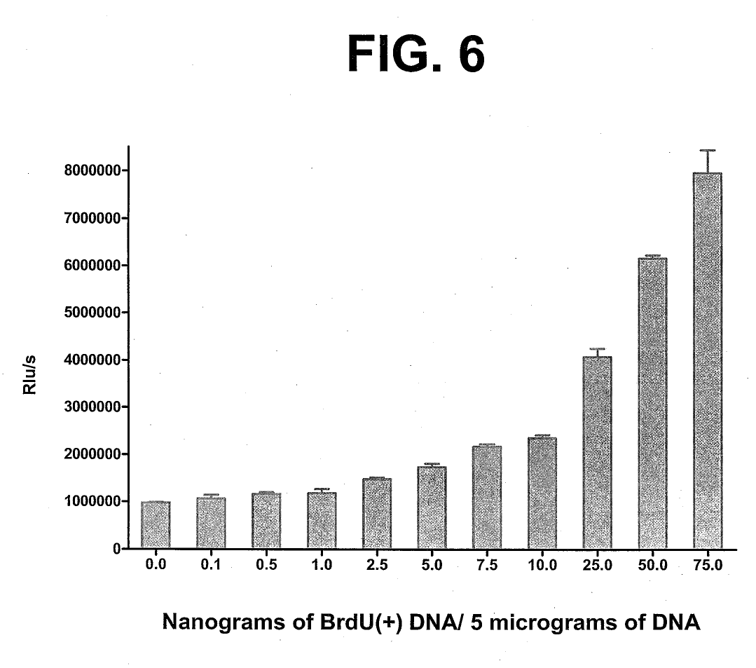

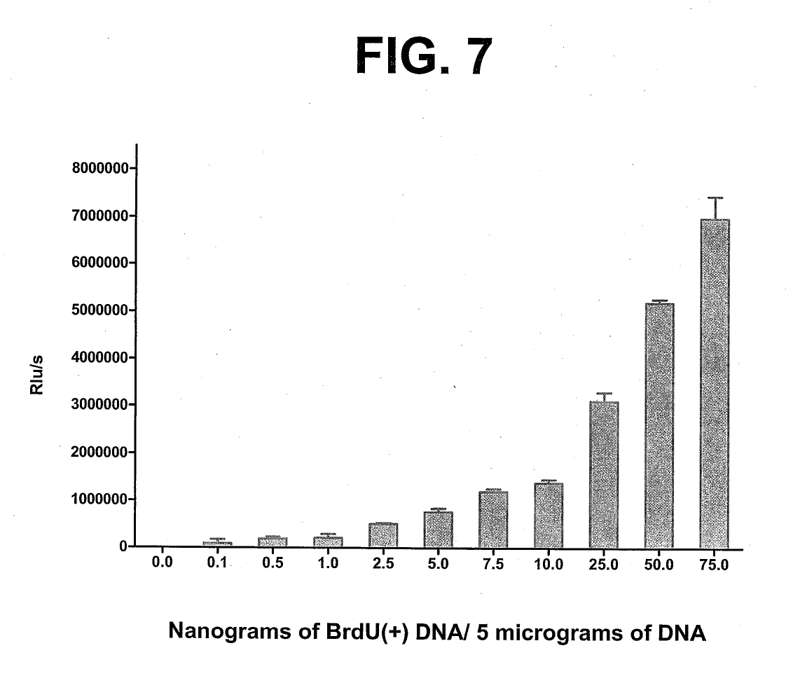

Results of a 1-step BrdU chemiluminescence ELISA with assay wells containing from 1 to 100 ng BrdU(+) DNA in 5 μg total DNA are shown in FIGS. 4 and 5 (the units in which chemiluminescence is quantified are relative light units (rlu) per second). FIG. 4 shows the measured (“raw”) chemiluminescence, and FIG. 5 shows the data after subtracting background (“bkg”) chemiluminescence determined for control assay wells containing no BrdU(+) DNA. FIGS. 6 and 7 show the results of a 1-step BrdU chemiluminescence ELISA with assay wells containing from 0.1 to 75 ng BrdU(+) DNA in 5 μg total DNA. FIG. 6 shows the raw chemiluminescence data, and FIG. 7 shows raw-bkg chemiluminescence. The relatively high background levels that were observed may be reduced by using a different type of surface plate. FIG. 7 clearly shows that the 1-step BrdU chemiluminescence ELISA can detect from 0.1 to 1 ng of BrdU(+) DNA in a total of 5 μg DNA. Assuming that 1 ng of DNA corresponds to about 100 cells, the 1-step BrdU chemiluminescence ELISA can detect BrdU(+) DNA of about 100 BrdU(+) cells in a total of about 500,000 cells.

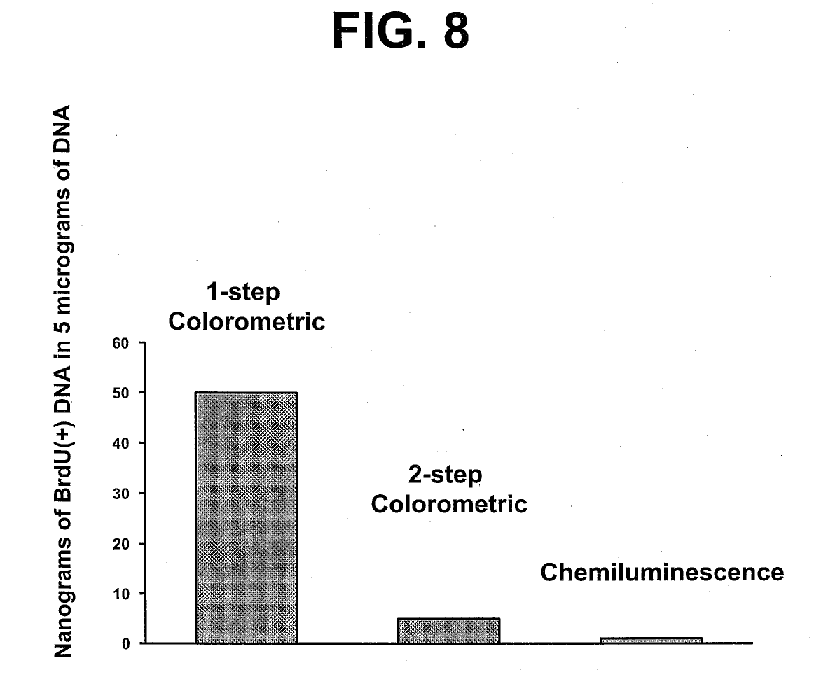

The relative sensitivities of the 1-step BrdU calorimetric ELISA, the 2-step BrdU calorimetric ELISA, and the 1-step BrdU chemiluminescence ELISA, are shown graphically in FIG. 8.

### Example 4

### Detection of Hippocampal Cell Proliferation in a Single Mammal Using a Chemiluminescence BrdU ELISA

This example demonstrates the use of chemiluminescence BrdU ELISA to detect BrdU incorporation into genomic DNA in the hippocampus of a single mammal.

### Subject Mammals

Male Sprague-Dawley rats (Charles River Laboratories, Inc., Wilmington, Mass.) weighing 200-225 g upon arrival into the animal facility were used in all studies described in this subsequent examples. The rats were housed in pairs, allowed access to food and water ad libitum, and maintained on a 12-h light-dark cycle, with lights on at 6 a.m. All injections were performed during the light cycle.

### BrdU Injection Protocols

Four different BrdU injection protocols were tested. Animals received ip. injections of 5-bromo-2-deoxyuridine (BrdU; Sigma, St. Louis, Mo., cat. no. B-5002) in saline (15 mg/ml) as follows:

(a) a single injection of 100 mg/kg BrdU ip. on one day,

(b) a single injection of 100 mg/kg BrdU ip. on each of 4 consecutive days,

(c) a single injection of 100 mg/kg BrdU ip. on each of 7 consecutive days, or

(d) four injections of 225 mg/kg BrdU ip. on one day, spaced two hours apart.

### Tissue Collection

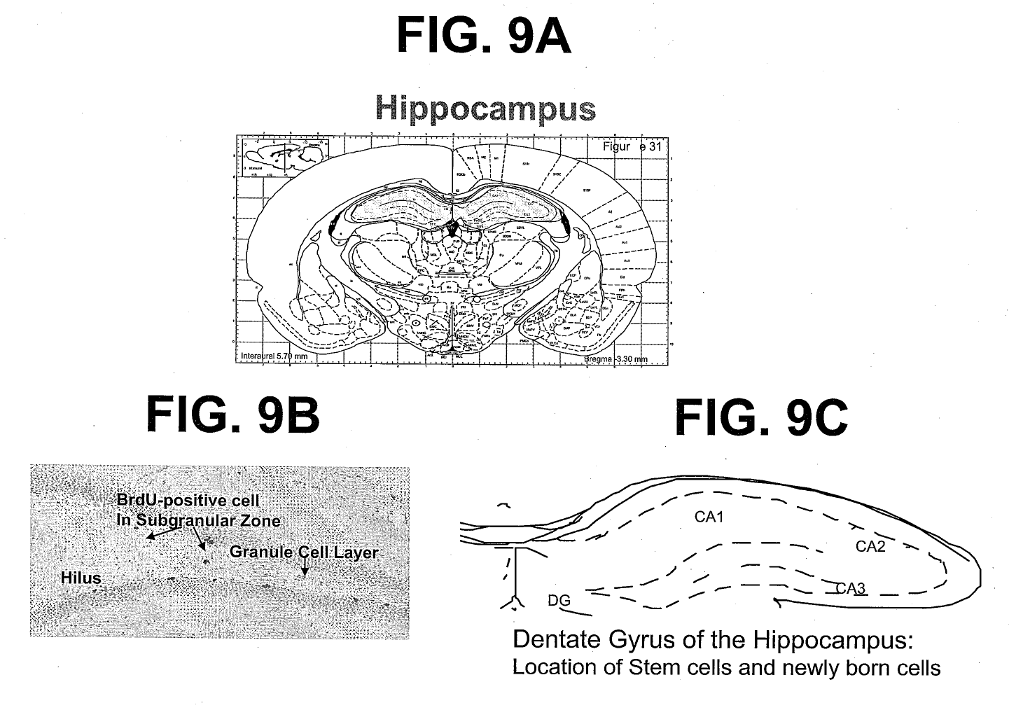

24 hours after the last BrdU injection, rats were euthanized with CO₂. The brains were extracted and placed in freezing saline. One person sectioned the brain tissue on a brain block (1.5-2 mm coronal sections), and a second person prepared at least three 1.5 mm punches of the subgranular zone of the dentate gyrus of the hippocampus (see FIGS. 9A-9C) taken from each animal. Samples were frozen immediately on dry ice, and were stored at −80° C. until DNA extraction.

Extraction of Genomic DNA from Hippocampus Tissue

Genomic DNA was extracted from cells of the samples using a DNEASY® 96 Tissue Kit (Qiagen, Valencia, Calif., cat. no. 69581), which provides a 96 well purification plate, the wells of which contain silica gel membranes that selectively bind DNA and allow contaminants to pass through. In accord with the kit protocol, the tissue samples were lysed by incubation overnight at 55° C. in buffered solution containing proteinase K, lysates were loaded into wells of the purification plate containing silica gel membranes, DNA bound to the membranes was washed, and purified DNA was eluted using buffer AE (10 mM Tris-Cl, 0.5 mM EDTA; pH 9.0) heated to 70° C.

BrdU-Containing DNA from Cultured Cells

BrdU incorporation into DNA of proliferating Chinese hamster ovary (CHO) cells served as a positive control for the ELISA experiments. CHO-KI cells were grown in Delbecco's Modified Eagle's Medium (DMEM) medium with 10% FBS in T 175 flasks and incubated with 100 μM BrdU for 48 hours. The cells were harvested and BrdU-containing genomic DNA was extracted and purified using a Cell Culture DNA Maxi Kit (Qiagen, cat. no. 13362) as described above.

### Detection of BrdU(+) DNA by Chemiluminescence BrdU ELISA

COSTAR® high binding 96 well plates (Corning Inc., Acton, Mass., cat. no. 3601) were coated with monoclonal anti-BrdU antibody (BD Biosciences, San Jose, Calif., cat. no. 347580) at a dilution of 1:100 (100 μl/well) in coating buffer (55 mM NaHCO₃ buffer; ph 9.0) overnight at 4° C. on a shaker. This was followed by blocking with SUPERBLOCK® high-protein blocking buffer (Pierce Biotechnology, Rockford, Ill.) for 60 minutes, and 5 washes with 1×TBST buffer (20 mM Tris, pH 7.8, 150 mM NaCl, 0.05% Tween-20) on a shaker at room temperature. 250 μl of buffer containing 5 μg of genomic DNA from a tissue sample was then applied to each well and incubated overnight at 4° C. on a shaker. The samples were washed three times with 1×PBS (137 mM NaCl, 2.7 mM KCl, and 10 mM phosphate buffer) on a shaker at room temperature. 100 μl of anti-BrdU peroxide (POD)-conjugated Fab antibody fragments (Roche Applied Science, Indianapolis, Ind., cat. no. 1585860) at a dilution of 1:100 was applied to each well and incubated overnight at 4° C. on a shaker. The wells were then washed 3 times with 1×PBS on a shaker at room temperature, and the amount of anti-BrdU POD-conjugated Fab antibody fragments bound to BrdU DNA in each well was quantified by determining the chemiluminescence produced upon addition of BM Chemiluminescence ELISA Substrate (POD) (Roche Applied Science, cat. no: 1 582 950) to the wells, following the manufacturer's instructions, and using a standard luminometer.

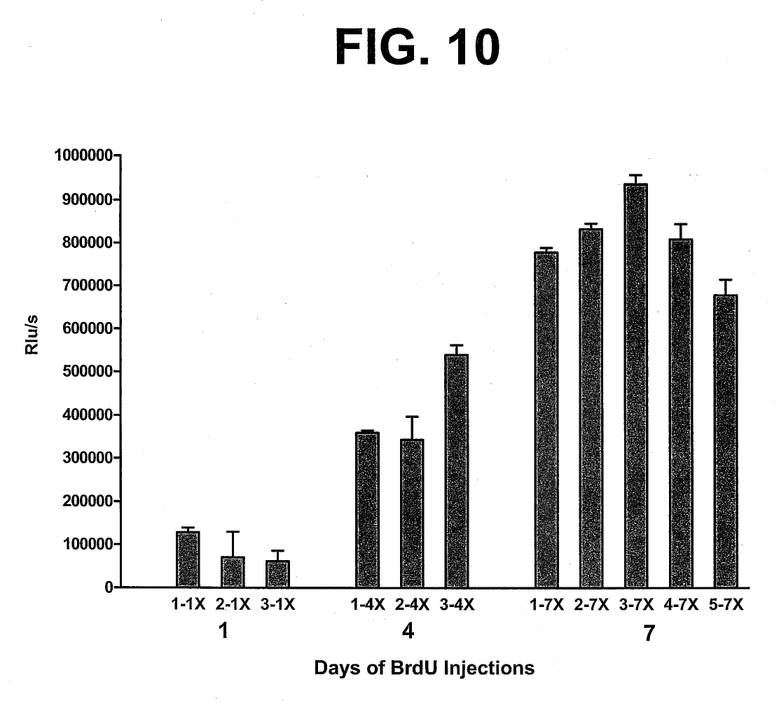

Results of the BrdU chemiluminescence ELISA for assay wells containing BrdU(+) DNA isolated from hippocampus of rats that received single, daily ip. injections of 100 mg/kg BrdU on one, four, or seven consecutive days are shown in FIG. 10. It is clear from FIG. 10 that the amount of chemiluminescence emitted in the assay, which is dependent on the amount of BrdU incorporated into the DNA, clearly increases as the number of days that BrdU is administered to the animals is increased from one to seven.

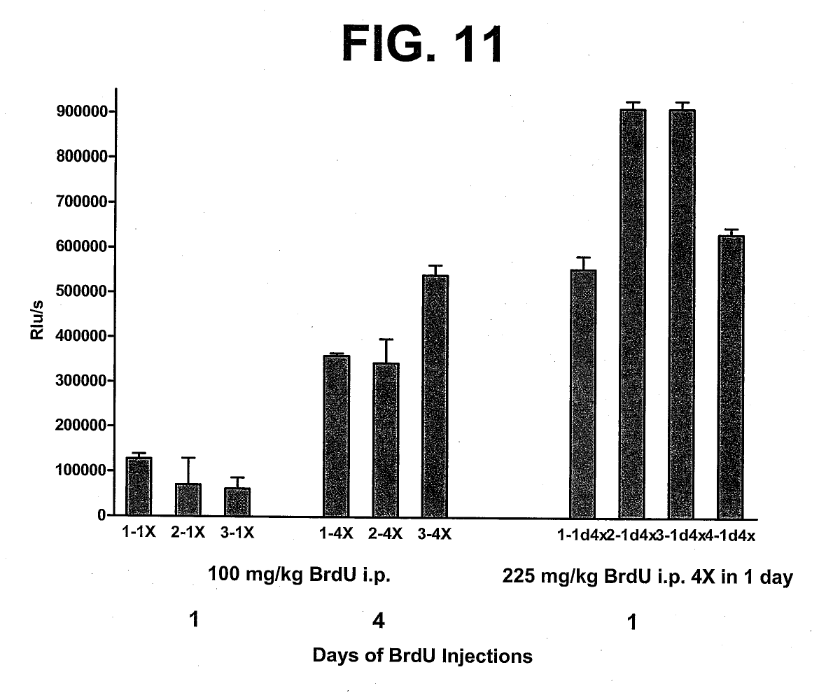

FIG. 11 shows the chemiluminescence determined in assay wells containing BrdU(+) DNA isolated from hippocampus of rats that received single, daily ip. injections of 100 mg/kg BrdU on one or four consecutive days, and from hippocampus of rats that received four ip. injections of 225 mg/kg BrdU on a single day, at 2-hour intervals. The results shown in FIG. 11 demonstrate that the BrdU chemiluminescence ELISA generates a relatively strong chemiluminescent signal upon detection of hippocampal BrdU(+) DNA of a single animal that was administered BrdU on a single day.

### Example 5

### Detecting Hippocampal Cell Proliferation in a Single Mammal Using Chemiluminescence BrdU ELISA Following Extraction of Genomic DNA from Whole Hippocampus

This example demonstrates the use of chemiluminescence BrdU ELISA to detect BrdU(+) DNA in genomic DNA extracted from whole hippocampus dissected from the brain of a single mammal.

### Subject Mammals

Male Sprague-Dawley rats (see Example 4) were used in this study.

### BrdU Injection Protocols

Each animal received four ip. injections of 225 mg/kg BrdU in saline on one day, spaced two hours apart.

### Tissue Collection

24 hours after the last BrdU injection, rats were euthanized with CO₂. Preparation of hippocampal punch samples was performed by a two-person team who sectioned brain tissue and took the punch samples, essentially as described in Example 4. Whole hippocampus dissection was performed by a single individual. Each brain was removed and immediately hemisected on ice, and the entire hippocampus was lifted out in one piece, in a procedure performed uniformly for each animal, and was placed immediately on dry ice. Samples were stored at −80° C. until DNA extraction. Individual hippocampus dissections consistently weighed 70-90 mg.

Extraction of Genomic DNA from Hippocampus Tissue

Genomic DNA was extracted from the samples in a uniform manner, essentially as described in Example 4. It was determined that genomic DNA can be efficiently extracted from cells of a dissected hippocampus by dividing the hippocampus into four parts of approximately the same mass, purifying the genomic DNA of each portion in a separate DNeasy column, and then pooling the wash solutions containing the genomic DNA from each hippocampus. By this method, approximately 40 μg of genomic DNA were extracted from each hippocampus.

### Detection of BrdU(+) DNA by Chemiluminescence BrdU ELISA

The samples were analyzed by chemiluminescence BrdU ELISA essentially as described in Example 4. Five μg of genomic DNA were applied to each assay well.

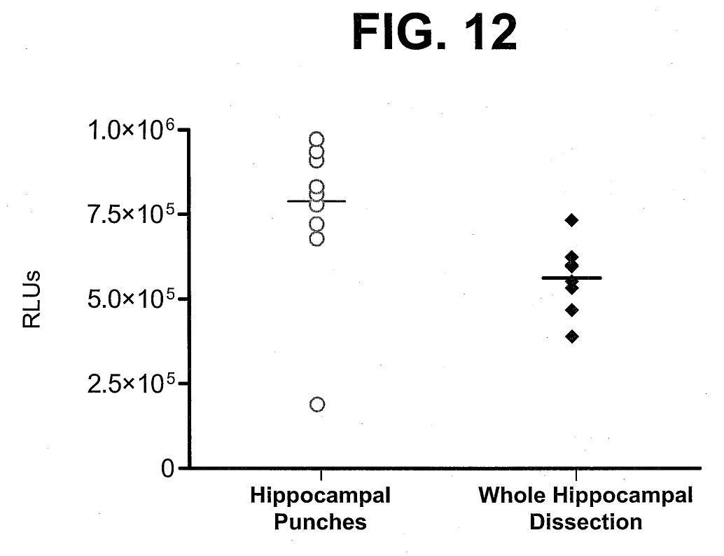

FIG. 12 shows the results of the BrdU chemiluminescence ELISA for assay wells containing BrdU(+) DNA isolated from hippocampus punch samples and from whole, dissected hippocampus. From FIG. 12 it can be seen that the BrdU chemiluminescence ELISA generates a relatively strong chemiluminescent signal indicating the presence of BrdU(+) DNA in genomic DNA extracted from the whole hippocampus of a single animal that was injected with BrdU on a single day.

### Example 6

### Dependence of BrdU Incorporation into Genomic DNA of Hippocampus Cells In Vivo on the Dosage of BrdU that is Administered

This example uses chemiluminescence BrdU ELISA to determine the relationship between the dosage of BrdU that is injected into an animal on a single day, and the amount of BrdU that is incorporated into genomic DNA of hippocampus cells of the animal in vivo in the 24 hour period following the last injection.

### Subject Mammals

Male Sprague-Dawley rats (see Example 4) were used in this study.

### BrdU Injection Protocols

Two experiments were performed. In one experiment, animals received four ip. injections of 25, 50, or 100 mg/kg BrdU in saline on one day, spaced two hours apart, or a single ip. injection of 200 mg/kg BrdU in saline on one day. In the other experiment, animals received four ip. injections of 100, 150, or 225 mg/kg BrdU in saline on one day, spaced two hours apart.

### Tissue Collection

24 hours after the last BrdU injection, rats were euthanized and whole hippocampus dissection was performed, essentially as described in Example 5.

Extraction of Genomic DNA from Hippocampus Tissue

Genomic DNA was extracted from the each of the whole hippocampus samples in a uniform manner, essentially as described in Example 5.

### Detection of BrdU(+) DNA by Chemiluminescence BrdU ELISA

The samples were analyzed by chemiluminescence BrdU ELISA essentially as described in Example 4. Ten μg of genomic DNA were applied to each assay well.

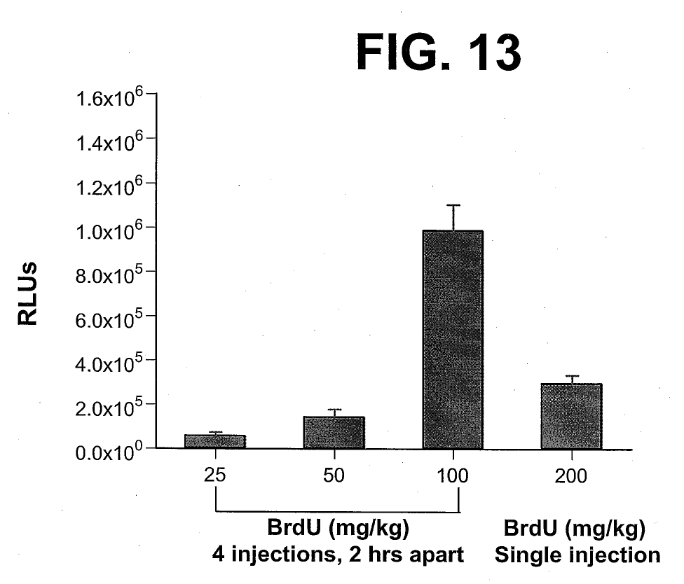

FIG. 13 shows the results of the BrdU chemiluminescence ELISA for assay wells containing BrdU(+) DNA extracted from whole hippocampus of animals that received on one day four ip. injections of 25, 50, or 100 mg/kg BrdU, or a single ip. injection of 200 mg/kg BrdU (experiment 1). FIG. 13 demonstrates that the amount of BrdU incorporated into hippocampal DNA following administration of BrdU on a single day depends on the total dosage of BrdU that is administered.

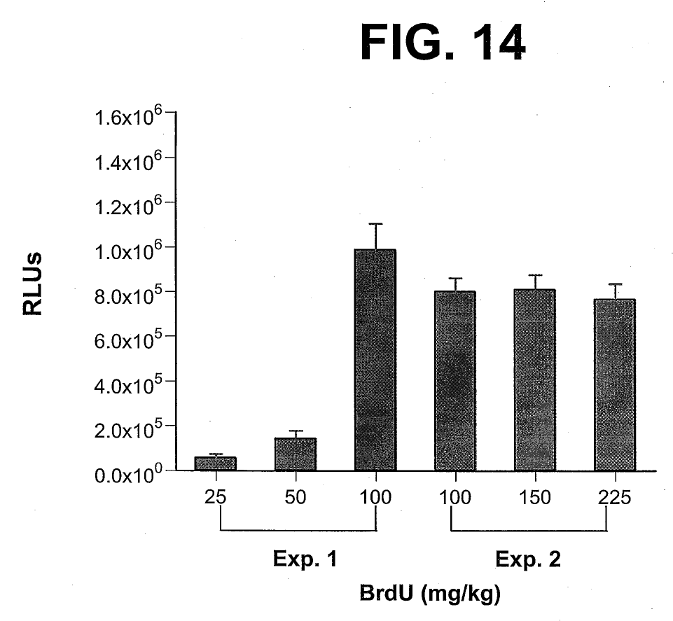

FIG. 14 shows the results of the BrdU chemiluminescence ELISA for assay wells containing BrdU(+) DNA extracted from whole hippocampus of animals that received on one day four ip. injections of 25, 50, or 100 mg/kg BrdU (experiment 1, also shown in FIG. 13), or four ip. injections of 100, 150, or 225 mg/kg BrdU (experiment 2). FIG. 14 demonstrates that following a regimen of four ip. injections given at two hour intervals on the same day, the amount of BrdU incorporated into hippocampal DNA is dose-dependent for dosages of up to 100 mg/kg BrdU, but that BrdU incorporation plateaus and does not increase further following injection of BrdU dosages greater than 100 mg/kg BrdU. In addition, it was observed that the variability of less than 10% of the chemiluminescent data for animals receiving doses of 100-225 mg/kg BrdU was similar to that which has been observed in manual counting experiments, whereas dosages of 25 and 50 mg/kg BrdU were associated with higher variability.

### Example 7

### Dependence of Signal Strength of Chemiluminescence BrdU ELISA on the Amount of Genomic DNA Applied to the Assay Well

This example demonstrates that increasing the amount of genomic DNA applied to each assay well from 5 to 10 μg/well results in an increase in strength of the chemiluminescent signal generated by chemiluminescence BrdU ELISA, without a significant increase in variability over that observed using 5 μg/well.

### Subject Mammals

Male Sprague-Dawley rats (see Example 4) were used in this study.

### BrdU Injection Protocols

All animals received four ip. injections of 100, 150, or 225 mg/kg BrdU in saline on one day, spaced two hours apart.

### Tissue Collection

24 hours after the last BrdU injection, rats were euthanized and whole hippocampus dissection was performed, essentially as described in Example 5.

Extraction of Genomic DNA from Hippocampus Tissue

Genomic DNA was extracted from the each of the whole hippocampus samples in a uniform manner, essentially as described in Example 5.

### Detection of BrdU(+) DNA by Chemiluminescence BrdU ELISA

The samples were analyzed by chemiluminescence BrdU ELISA essentially as described in Example 4. Aliquots of 5 μg and 10 μg of each genomic DNA sample were analyzed in separate assay wells in duplicate or triplicate, depending on the amount of DNA that was available.

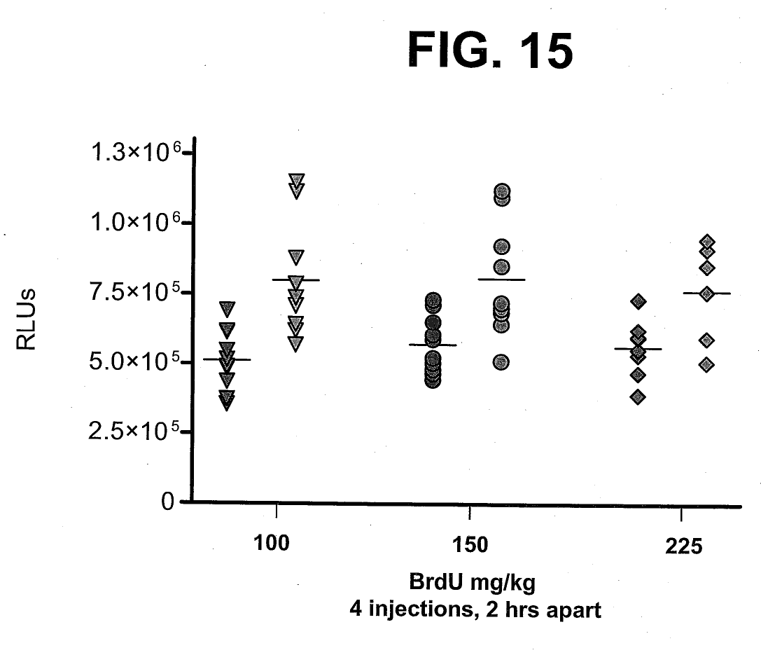

FIG. 15 shows the results of the BrdU chemiluminescence ELISA for assay wells that received 5 μg or 10 μg of genomic DNA extracted from whole hippocampus of animals that received on one day four ip. injections of 100, 150, or 225 mg/kg BrdU. FIG. 15 demonstrates that increasing the amount of genomic DNA applied to the assay well from 5 to 10 μg/well provides a significant increase in strength of the chemiluminescent signal generated by chemiluminescence BrdU ELISA, without greatly increasing the variability of the data.

### Example 8

### Detecting ECS-Induced Increases in BrdU Incorporation into Hippocampal DNA of Individual Mammals Using Chemiluminescence BrdU ELISA

This example demonstrates that chemiluminescence BrdU ELISA can be used to detect and quantify ECS-induced increases in BrdU incorporation into hippocampal DNA of an individual laboratory mammal (e.g., rat).

### Subject Mammals

Male Sprague-Dawley rats (see Example 4) were used in this study.

### Electroconvulsive Shock (ECS)

Rats were administered ECS once daily for 10 days. To administer the ECS, the rat was lightly restrained by being wrapped in a paper towel, with its head exposed. Conducting jelly was applied to the ears and a single ECS was administered via earclip electrodes (50 mA, 0.5 seconds). This level and duration of current produces a seizure that lasts less than 1 min and is characterized by full extension of the hind limbs (tonic phase) for 10-15 sec., followed by repetitive flexion-extension of the forelimbs (clonic phase) for 10-15 sec. After the cessation of the shock, each rat was placed in a plastic cage where it remained singly housed for one hour, at which point it was returned to its home cage. Earclips were applied to sham control animals, but no electrical current was administered.

### Bromodeoxyuridine Injections

24 hours after the last ECS treatment, all animals received four consecutive ip. injections of 100 or 150 mg/kg BrdU in saline on one day, spaced two hours apart.

### Hippocampal Dissection

24 hours after the last BrdU injection, the rats were euthanized and whole hippocampus dissection was performed, essentially as described in Example 5.

Extraction of Genomic DNA from Hippocampus Tissue

Genomic DNA was extracted from the each of the whole hippocampus samples in a uniform manner, essentially as described in Example 5.

### Detection of BrdU(+) DNA by Chemiluminescence BrdU ELISA

The samples were analyzed by chemiluminescence BrdU ELISA essentially as described in Example 4. Aliquots of 5 μg and 1° μg of each genomic DNA sample were analyzed in separate assay wells.

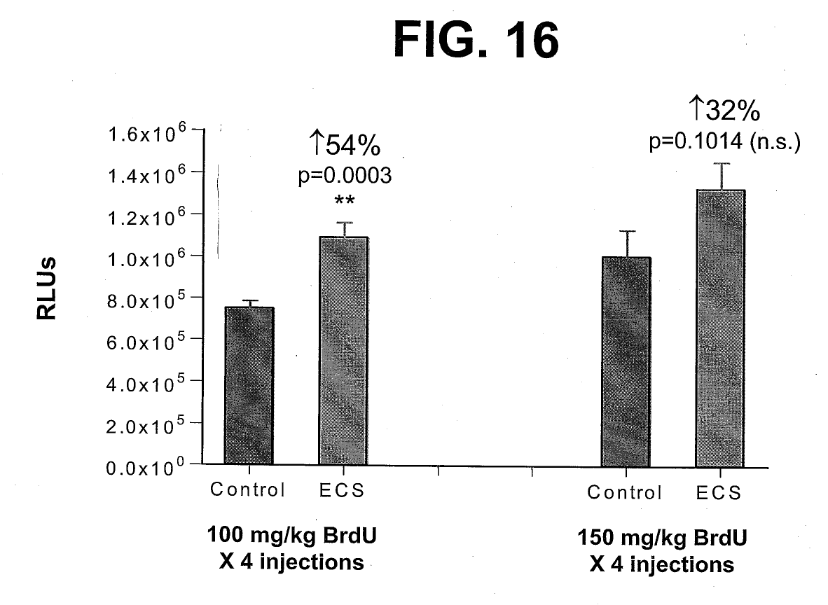

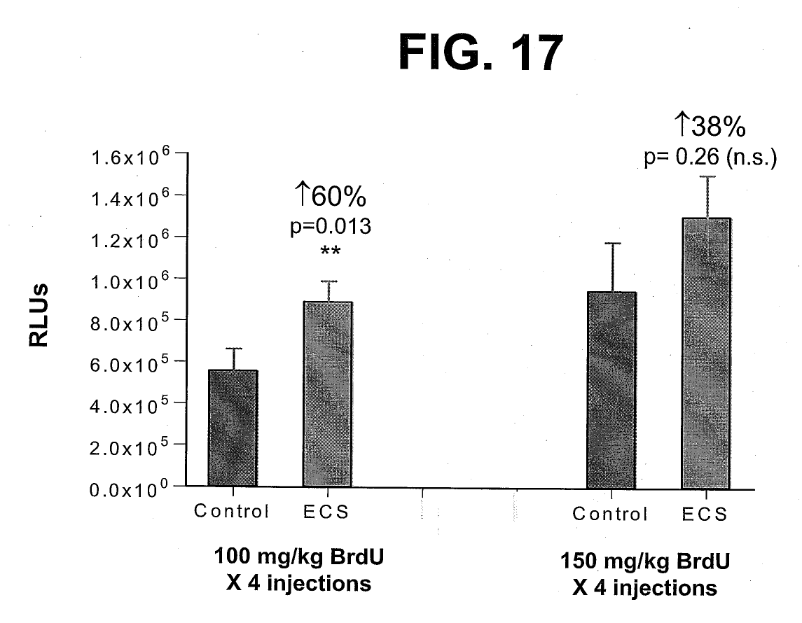

FIGS. 16 and 17 show the results of the BrdU chemiluminescence ELISA for assay wells containing BrdU(+) DNA extracted from whole hippocampus of animals that received four ip. injections of 100 or 150 mg/kg BrdU on one day following ECS treatment. FIGS. 16 and 17 demonstrate that BrdU chemiluminescence ELISA detects a greater than 50% increase in BrdU incorporation in the hippocampus of individual rats given 10 days of ECS over the level of BrdU incorporation in control animals that did not receive ECS. BrdU chemiluminescence ELISA results obtained using 10 μg DNA/well from rats that received 4 injections of 100 mg/kg BrdU in one day showed <10% variability. Greater variability was observed for data for rats that received 4 injections of 150 mg/kg BrdU in one day. Manual BrdU counting experiments have reported a ˜50% increase in BrdU-positive cells in the dentate gyrus after chronic ECS. The increase detected by BrdU chemiluminescence ELISA is thus similar to that seen with manual cell counting experiments.

Those skilled in the art will recognize, or be able to ascertain using no more than routine experimentation, many equivalents of the specific examples of the invention described herein. Such equivalents are intended to be encompassed by the following claims.

What is claimed is:

1. A method of detecting and quantifying cell proliferation in a mammal, comprising the steps of:
(a) obtaining genomic DNA from cells of a hippocampal tissue sample, which sample was obtained from a mammal having received an amount of 5-halo-2′-deoxyuridine (HdU) sufficient to effect incorporation of HdU into newly synthesized DNA of proliferating cells in the mammal;
(b) contacting the genomic DNA of step (a) with monoclonal anti-HdU antibodies under conditions in which the antibodies bind to HdU-containing DNA, and wherein an enzyme that catalyzes conversion of a substrate to a light-producing product is attached to the anti-HdU antibodies that are bound to the HdU-containing DNA;
(c) contacting the anti-HdU antibodies that are bound to the HdU-containing DNA with a solution containing a substrate compound that is converted to a light-emitting product by the enzyme of step (b), at a sufficient substrate concentration and under conditions such that a chemiluminescent signal is emitted; and
(d) determining the intensity of the chemiluminescent signal emitted in step (c).

2. The method of claim 1, wherein the mammal is a non-human mammal selected from the group consisting of mouse, rat, cat, dog, pig, and non-human primate.

3. The method of claim 1, wherein said sample was obtained from a mammal treated with a physical, chemical, dietary, or pharmacological treatment that stimulates or inhibits cell proliferation in the tissue of interest of the mammal prior to receiving the HdU.

4. The method of claim 1, wherein the tissue sample comprises at least one tissue selected from the group consisting of blood, bone marrow, pancreas, liver, kidney, intestines, thymus, heart, striated muscle, spinal cord, and brain.

5. The method of claim 4, wherein the tissue sample comprises at least one brain tissue selected from the group consisting of hippocampus and subventricular zone adjacent to lateral ventricles.

6. The method of claim 1, wherein the HdU of step (a) is 5-bromo-2′-deoxyuridine (BrdU), and the antibodies of step (b) are monoclonal anti-BrdU antibodies that bind to BrdU-containing DNA.

7. The method of claim 6, wherein the sample of step (a) was obtained from a mammal having received from 25 to 1500 mg/kg BrdU in one day.

8. The method of claim 1, wherein step (b) further comprises contacting the genomic DNA with a support material that binds to HdU-containing DNA, under conditions in which the HdU-containing DNA binds to the support material.

9. The method of claim 1, wherein the enzyme of step (b) is selected from the group consisting of peroxidase, alkaline phosphatase, β-galactosidase, acid phosphatase, and β-glucuronidase.

10. A method of detecting and quantifying cell proliferation in the hippocampus of a mammal, comprising the steps of:
(a) obtaining genomic DNA from cells of a hippocampal tissue sample, which sample was obtained from a non-human mammal having received an amount of 5-bromo-2′-deoxyuridine (BrdU) sufficient to effect incorporation of BrdU into newly synthesized DNA of proliferating cells in the hippocampus of the mammal;
(b) contacting the genomic DNA of step (a) with monoclonal anti-BrdU antibodies under conditions in which the antibodies bind to BrdU-containing DNA, and wherein an enzyme that catalyzes conversion of a substrate to a light-producing product is attached to the anti-BrdU antibodies that are bound to the BrdU-containing DNA;
(c) contacting the anti-BrdU antibodies that are bound to the BrdU-containing DNA with a solution containing a substrate compound that is converted to a light-emitting product by the enzyme of step (b), at a sufficient substrate concentration and under conditions such that a chemiluminescent signal is emitted; and
(d) determining the intensity of the chemiluminescent signal emitted in step (c).

11. The method of claim 10, wherein the mammal is a non-human mammal selected from the group consisting of mouse, rat, cat, dog, pig, and non-human primate.

12. The method of claim 10, wherein the sample of step (a) was obtained from a mammal having received from 25 to 1500 mg/kg BrdU in one day.

13. The method of claim 12, wherein the mammal received BrdU by intraperitoneal injection.

14. The method of claim 10, wherein said sample was obtained from a mammal treated with a physical, chemical, dietary, or pharmacological treatment that stimulates or inhibits cell proliferation in the tissue of interest prior to receiving the 5-bromo-2′-deoxyuridine (BrdU).

15. The method of claim 10, wherein the enzyme of step (b) is selected from the group consisting of peroxidase, alkaline phosphatase, β-galactosidase, acid phosphatase, and β-glucuronidase.

16. A method of determining if a physical, chemical, dietary, or pharmacological treatment of interest stimulates or inhibits cell proliferation in a tissue of a mammal comprising:
(a) treating the mammal with the treatment of interest;
(b) administering an amount of a 5-halo-2′-deoxyuridine (HdU) to the mammal sufficient to effect incorporation of HdU into newly synthesized DNA of proliferating cells in the mammal, wherein the administering of an amount of HdU is performed simultaneously or sequentially with the treating of step (a);
(c) removing a tissue sample from the mammal;
(d) extracting genomic DNA from cells of the tissue sample;
(e) contacting the genomic DNA of step (d) with monoclonal anti-HdU antibodies under conditions in which the antibodies bind to HdU-containing DNA, and an enzyme that catalyzes conversion of a substrate to a light-producing product is attached to the anti-HdU antibodies that are bound to the HdU-containing DNA;
(f) contacting the anti-HdU antibodies that are bound to the HdU-containing DNA with a solution containing a substrate compound that is converted to a light-emitting product by the enzyme of step (e), at a sufficient substrate concentration and under conditions such that a chemiluminescent signal is emitted;
(g) determining the intensity of the chemiluminescent signal emitted in step (f) to detect and quantify the amount of HdU incorporated into the DNA; and
(h) comparing the amount of HdU incorporated into the DNA with the amount of HdU incorporated into DNA extracted from the same type of tissue of a control mammal that has not received the treatment of interest, as determined by steps (b)-(g), to determine if the treatment stimulates or inhibits cell proliferation in the tissue.

17. The method of claim 16, wherein the treatment of interest of step (a) comprises administering a compound to the mammal, and step (h) comprises comparing the amount of HdU incorporated into the DNA with the amount of HdU incorporated into the DNA extracted from the same tissue of a control mammal to which the compound has not been administered as determined by steps (b)-(g) to determine if the compound stimulates or inhibits cell proliferation in the tissue.

18. The method of claim 16, wherein the HdU is BrdU, and step (b) comprises administering from 25 to 1500 mg/kg BrdU to the mammal in one day.

19. The method of claim 16, wherein the tissue sample comprises at least one tissue selected from the group consisting of blood, bone marrow, pancreas, liver, kidney, intestines, thymus, heart, striated muscle, spinal cord, and brain.

20. The method of claim 16, wherein the tissue sample comprises at least part of a tissue sample obtained from hippocampus.
